(19) United States
(12) Patent Application Publication
(10) Pub. No.: US 2017/0087008 A1
(43) Pub. Date: Mar. 30, 2017

(54) ELONGATED COOLING GEL BAND

(71) Applicant: William Gideon ALSTON, Opelika, AL (US)

(72) Inventor: William Gideon ALSTON, Opelika, AL (US)

(21) Appl. No.: 15/157,960

(22) Filed: May 18, 2016

Related U.S. Application Data
(60) Provisional application No. 62/232,069, filed on Sep. 24, 2015.

(51) Int. Cl. A61F 7/02

(52) U.S. Cl. CPC A61F 7/02; A61F 2007/0007

(57) ABSTRACT

An elongated gel band is provided. The elongated gel band is dimensioned so that a gel-filled tubing sheathed by a covering sleeve is adapted to bend around various body parts, providing cooling temperatures for 45 to 60 minutes. The covering sleeve is adapted to removably connect opposing ends thereof so as to secure the elongated gel band in a desired place. The elongated gel band can be made in various lengths to accommodate various head or neck sizes. Extra long elongated gel bands (36″ to 50″) will provide immediate and highly effective treatment for injuries to various body parts, especially joints.

### CROSS-REFERENCE TO RELATED APPLICATION

This application claims the benefit of priority of U.S. provisional application No. 62/232,069, filed 24-Sep.-2015, the contents of which are herein incorporated by reference.

### BACKGROUND OF THE INVENTION

The present invention relates to ice and cooling packs and, more particularly, an elongated cooling gel band for selectively securing to various body parts, and embodying methods for using the same for preventing overheating and treating injuries.

Traditional cooling devices, such as ice packs, are not adapted and dimensioned for selectively securing to various portions of a user's body in the form of an elongated band. Current cooling devices, such as wet-and-snap cooling fabrics (cloths) are ineffective, typically providing less than ten minutes of cooling, before requiring rewetting.

As can be seen, there is a need for an elongated cooling gel band for selectively securing to various body parts, embodying methods for preventing overheating by being disposed in close contact with major arteries in the head and/or neck so as to reduce the individual's body temperature, which in turn reduces sweating and provides overall cooling comfort. There is also a need for an elongated cooling gel band for treating injuries. The elongated band can be easily wrapped and tied around an injured joint or area so that the area is completely encircled. Standard gel packs must be secured with tape and only treat one side of the injured area. Moreover, the elongated cooling gel bands permit greater mobility for the patient during treatment.

### SUMMARY OF THE INVENTION

In all aspects of the present invention, an elongated cooling gel band includes an elongated tubing filled with a cooling gel, wherein the cooling gel is adapted to be pliable when frozen. The elongated cooling gel band includes the elongated tubing with a length to width ratio of approximately 12:1 for use as a head/neck band and a length to width ratio of up to 26:1 for injury-treatment bands and further includes a covering sleeve dimensioned to slidably receive the entire elongated tubing. The elongated cooling gel band may further include a cooperating connector disposed on each of the opposing ends of the covering sleeve, or may be extend substantially longer than the tubing length so that the opposing ends of the covering sleeve may be tied together. Moreover, the covering sleeve provides an opening for inserting and removing the elongated tubing. The cooling gel tubing is filled with a formula made up of water, salt, and a starch.

### BRIEF DESCRIPTION OF THE DRAWINGS

FIG. 1 is a perspective view of an exemplary embodiment of the present invention, shown in use;

FIG. 2 is a detailed perspective view of an exemplary embodiment of the present invention, shown in use;

FIG. 3 is a perspective view of an exemplary embodiment of the present invention, shown in use;

FIG. 4 is a detailed perspective view of an exemplary embodiment of the present invention, shown in use;

FIG. 5 is a perspective view of an exemplary embodiment of the present invention, shown in use as an injury-treatment band;

FIG. 6 is a detailed perspective view of an exemplary embodiment of the present invention, shown in use as an injury-treatment band;

FIG. 7 is a perspective view of an exemplary embodiment of the present invention, shown with a detachable connector;

FIG. 8 is a section detail view of an exemplary embodiment of the present invention, taken along line 8-8 of FIG. 7;

FIG. 9 is a section detail view of an exemplary embodiment of the present invention, taken along line 9-9 of FIG. 7;

FIG. 10 is a perspective view of an exemplary embodiment of the present invention, shown with a knot tie; and

FIG. 11 is a section detail view of an exemplary embodiment of the present invention, illustrating an opening in the covering sleeve for insertion/removal of gel tubing.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides an elongated gel band providing a gel-filled tubing sheathed by a covering sleeve, wherein the covering sleeve is adapted to removably connect opposing ends thereof so that the gel tubing, which is adapted to bend around various body parts, can provide cooling temperatures for 45 to 60 minutes.

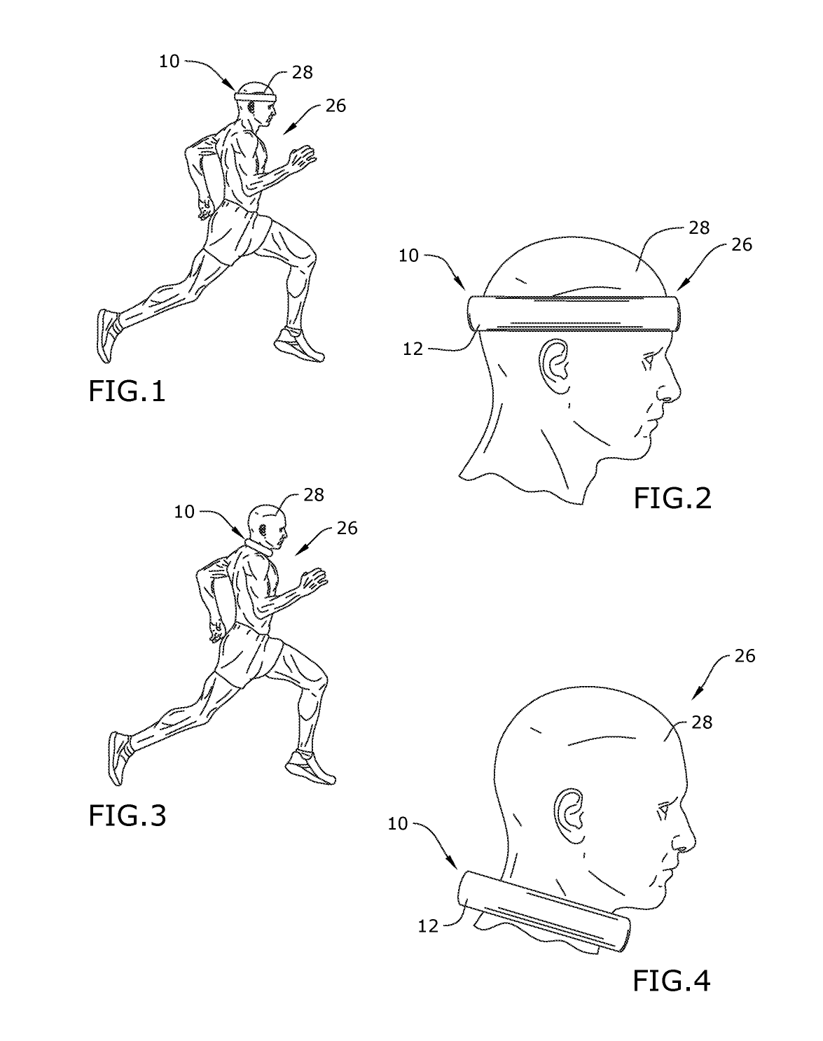

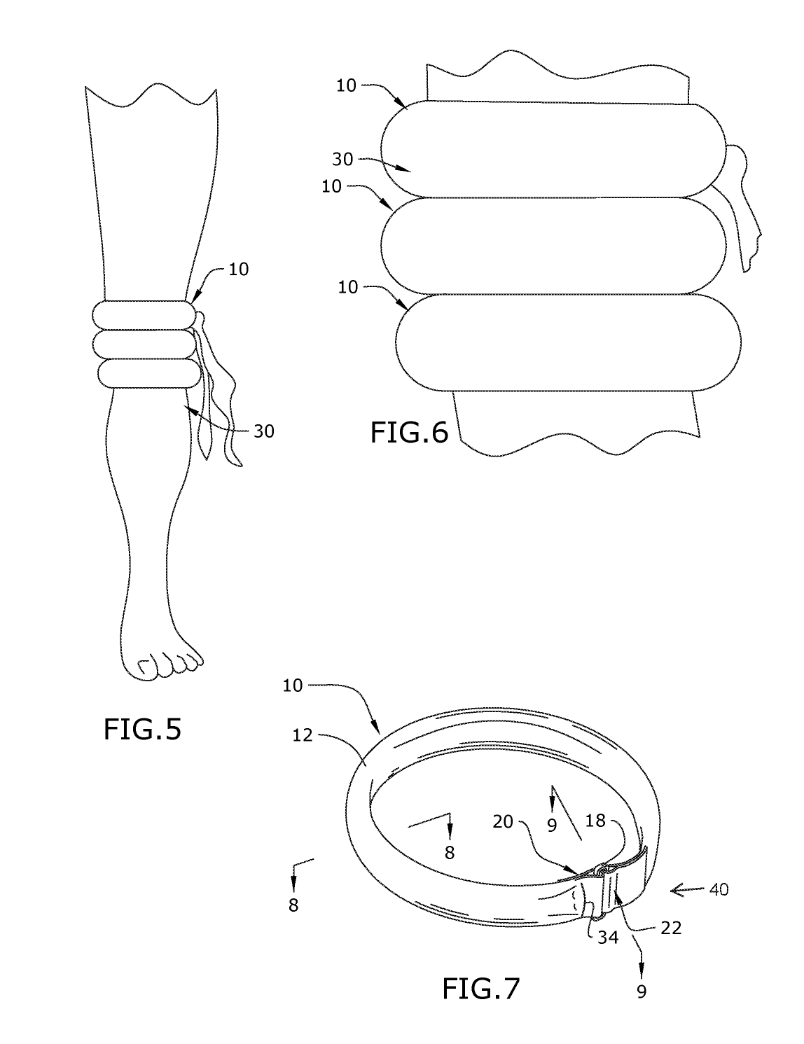

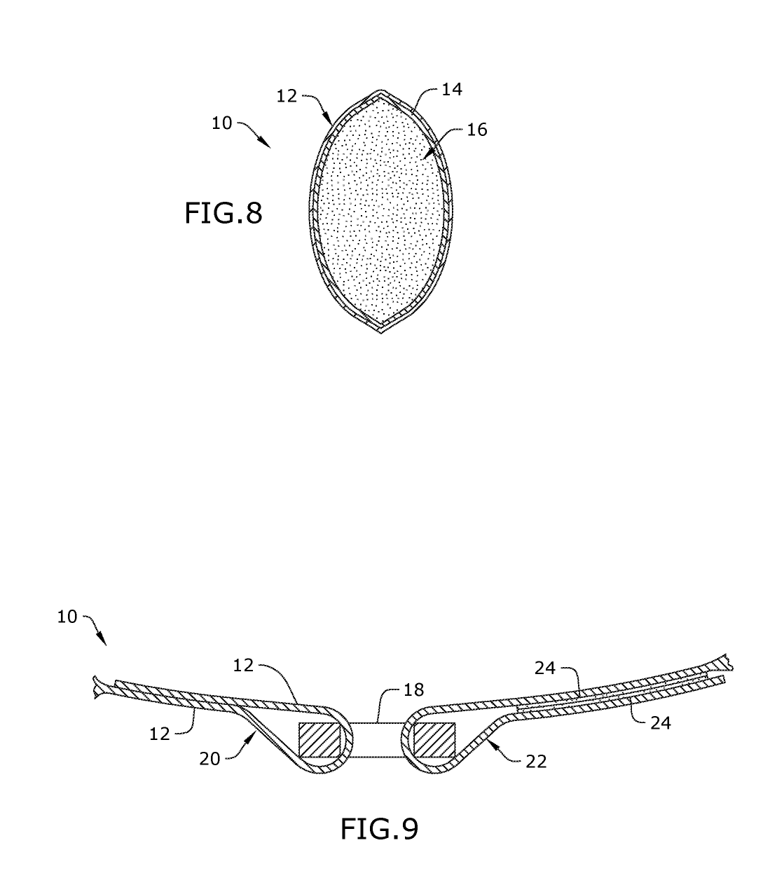

Referring now to FIGS. 1 through 11, the present invention may include an elongated gel band assembly 10. The elongated gel band assembly 10 may include a gel formula 16, a gel tubing 14 and a covering sleeve 12.

The gel formula 16 may have freezing and/or cooling properties as a function of its chemical composition. The chemical composition may be adapted to include at least water, salt and a starch or other chemicals so as to maintain a first predetermined viscosity at room temperature and a second predetermined viscosity when subject to temperatures around 32 degrees Fahrenheit. Moreover, the chemical composition may be adapted so that the second predetermined viscosity provides a non-rigid pliable physical property so as to facility its physical conformity to the curves and contours of different human wearers. The chemical composition may be adapted so that the gel formula 16 maintains temperatures around 32-50 degrees Fahrenheit for approximately 45 to 60 minutes.

The gel tubing 14 may be made of flexible, temperature-insulating material(s) that can be bent and twisted repeatedly without failure even when subject to sub-freezing temperatures, such as various plasticized materials, like polycarbonate.

The covering sleeve 12 may be made of any comfortable, resilient material, such as cotton or blends thereof, so as to be repeatedly stretched without failure, even when subject to freezing temperatures. The covering sleeve 12 extends from a first end 42 to a second end 44, forming a lumen throughout. One end, for example the second end 44, may be sealed.

A connector or connectors 40 may be disposed near each end 42, 44. The connector(s) 40, in certain embodiments, may include a clip 18 secured within a fixed clip loop 20 connected to the first end 42, wherein a hook-and-loop fastener 24 may be disposed on the opposing second end 44 so that the second end can be formed into an adjustable loop 22 for removably securing the opposing clip 18, as illustrated in FIGS. 7 and 9. It should be understood that the connector(s), in selective embodiments, may be any fastener known in the art for fastening or removably securing one object to another including, for example, standard push-button snaps, hook-and-loop-type fasteners, adhesive substances, combinations thereof, and the like. It should also be that the connector(s) 40 may be configured in any array and/or number, so long as the connector(s) 40 function in accordance with the present invention as described herein.

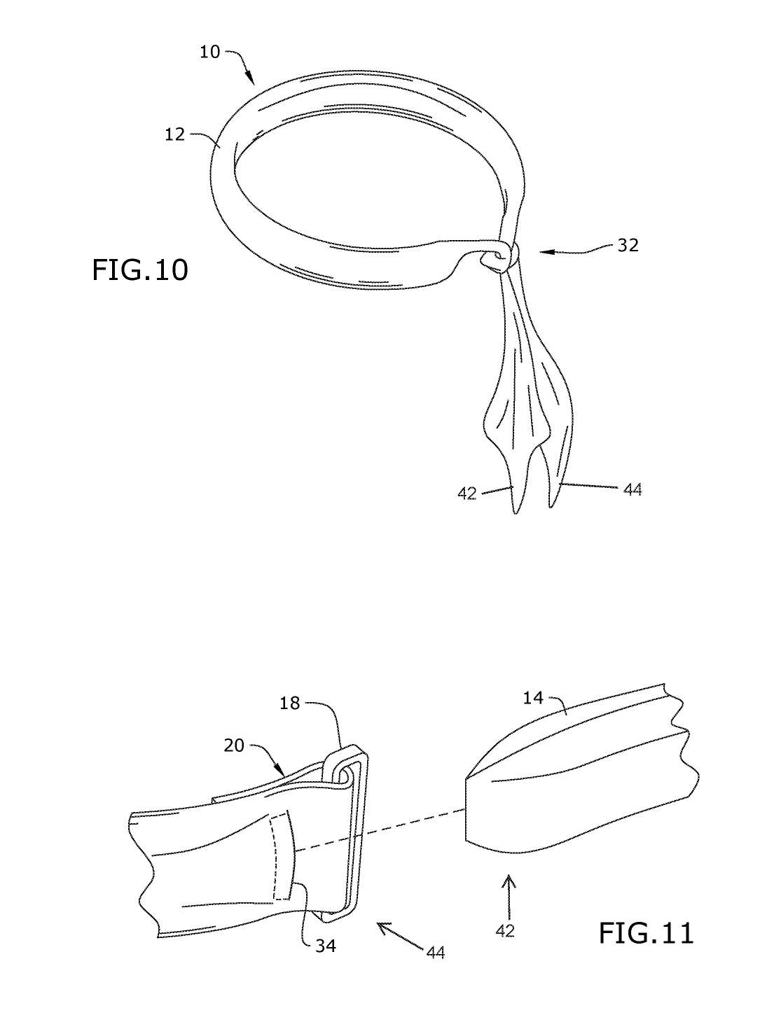

In certain embodiments, the gel tubing 14 lumen may be approximately 2 inches in diameter and have a length of about 24 inches, providing an elongated shape at approximately a 12:1 ratio of length to width. The lumen of the covering sleeve may be dimensioned and adapted to slidably receive the gel tubing 14 substantially filed with gel formula 16. In certain embodiments, the first end 42 and the second end 44 of the covering sleeve 12 each extends substantially beyond the length of the gel tubing, so that said first and second ends 44 may be tied together, forming a knot 32 so as to secure it to a user's body part, as illustrated in FIG. 10.

A process of making the present invention may include the following. A maker may cut an approximately 2 inch, 6 mil gel tubing 14 to fit a desired head or body part size and seal one end. Then the maker may inject the gel formula 16 into the gel tubing 14 until it's about 80% full, leaving about, in certain embodiments, 4 inches of empty space in certain embodiments. Then the maker may squeeze the air from that space within the lumen, and seal the open end. Knead the gel tubing 14 until the gel formula 16 is evenly distributed within the gel tubing 14.

In certain embodiments, the long injection or extrusion valve may inject the gel formula 16 into the gel tubing 14 from bottom to top so as to avoid getting any gel formula 16 on the last 2 inches of the open gel tubing 14. The gel tubing 14 end must be cleaned and dry for proper sealing.

Then the maker may make a fabric by sewing one side to produce the tubular covering sleeve 12 that may be approximately 2½ inches wide and 23 to 26 inches in length (depending on desired head size). Dry-Fit fabric works well for this. Reverse (turn) the covering sleeve 12 so that the stitches are on the inside. Sew or clamp a connector(s) 40 to one or both of the first and second ends 42, 44 of the covering sleeve 12.

The maker may slide the gel-formula-16-filled gel tubing 14 through an opening 34 provided by the covering sleeve 12, whereby the gel tubing 14 can be removed later and the band can be washed.

A method of using the present invention may include the following. The elongated gel band assembly 10 disclosed above may be provided. To use the product, a user may simply place the elongated gel band assembly 10 or the gel-formula-16-filled gel tubing 14 in a freezer overnight. The gel formula 16 in the gel tubing 14 freezes completely but remains soft and pliable. If several elongated gel bands 10 are kept in a cooler, they can provide many hours of comfort and protection. The gel-formula-16-filled gel tubing 14 slides neatly into the covering sleeve 12 and stays there through multiple uses. The band can be refrozen and reused hundreds of times. The gel tubing 14 can be easily removed for washing of the covering sleeve 12.

The frozen elongated gel band assembly 10 remains soft/pliable and can be tied or buckled (via the connector 40) around the head 28, the neck, or the knee 30 of a user 26, as illustrated in FIGS. 2, 4 and 5, respectively. The frozen band can also be used by tying or buckling it around the neck. Essentially, via the elongated gel band assembly 10, the frozen gel formula 16 encircling the head prevents forehead sweat and cooling the entire body, whereby the covering sleeve may absorb or wick any perspiration of condensation. The cooling effects of one elongated gel band assembly 10 may last 45 to 60 minutes, whereby multiple elongated gel bands 10 stored in a cooler can give all-day relief and protection. As a result, the elongated gel band assembly 10 can replace or assist the cooling and moistening fans seen on football fields. They can also keep sweat out of the eyes of athletes and make the hottest days comfortable.

It should be understood, of course, that the foregoing relates to exemplary embodiments of the invention and that modifications may be made without departing from the spirit and scope of the present invention.

Any elongated gel band can be effectively used for both general cooling and injury treatment, but doctors, athletic trainers, and personal trainers will want longer bands (36″ to 50″) in order to completely encircle injured knees, elbows, shoulders, hips, and other areas of the body.

What is claimed is:

1. A device, comprising:
an elongated tubing filled with a cooling gel, wherein the cooling gel is adapted to be pliable when frozen.

2. The device of claim 1, wherein the elongated tubing has a tubing length to a tubing width ratio of approximately 12:1.

3. The device of claim 2, further comprising a covering sleeve dimensioned to slidably receive the entire elongated tubing.

4. The device of claim 3, further comprising a cooperating connector disposed on each of the opposing ends of the covering sleeve.

5. The device of claim 3, wherein the covering sleeve has a sleeve length extending substantially longer than the tubing length so that the opposing ends of the covering sleeve may be tied together.

6. The device of claim 3, wherein the covering sleeve provides an opening for sliding the elongated tubing there through.

7. The device of claim 1, wherein the cooling gel comprises water, salt and a starch.
